(19) United States
(12) Patent Application Publication
(10) Pub. No.: US 2015/0320892 A1
(43) Pub. Date: Nov. 12, 2015

(54) IMAGING FIBROSIS

(71) Applicant: GE Healthcare Limited, Buckinghamshire (GB)

(72) Inventors: Andreas OLSSON, Oslo (NO); Radha ACHANATH, Bangalore (IN); Peter Brian IVESON, Amersham (GB); David TUCH, Chesham (GB)

(21) Appl. No.: 14/410,908

(22) PCT Filed: Jun. 28, 2013
(86) PCT No.: PCT/EP2013/063702

§ 371 Date: Dec. 23, 2014

Related U.S. Application Data
(60) Provisional application No. 61/701,759, filed on Sep. 17, 2012.

(30) Foreign Application Priority Data
Jun. 29, 2012 (IN) 2047/DEL/2012
Sep. 17, 2012 (GB) 1216530.4

(51) Int. Cl. A61K 51/04; C07F 5/02

(52) U.S. Cl. CPC A61K 51/0478; C07F 5/025; A61K 2123/00

(57) ABSTRACT

The present invention provides a labelled compound suitable for use as an in vivo imaging agent. The in vivo imaging agent of the invention is useful in the in vivo diagnosis and imaging of fibrosis and in particular fibrosis in the liver. Also provided by the present invention is a method for the preparation of the labelled compound of the invention and a precursor compound useful in said method and a kit useful for carrying out said method. In addition, the present invention provides a pharmaceutical composition comprising the labelled compound of the invention as well as a method of in vivo imaging using the labelled compound of the invention, preferably as the pharmaceutical composition of the invention.

### CROSS-REFERENCE TO RELATED APPLICATIONS

This is a national stage application under 35 U.S.C. §371(c) of prior-filed, co-pending, PCT application serial number PCT/EP2013/063702, filed on Jun. 28, 2013, which claims priority to Indian patent application serial number 2047/DEL/2012, filed on Jun. 29, 2012, British patent application serial number 1216530.4, filed on Sep. 17, 2012, and U.S. provisional patent application Ser. No. 61/701,759, filed on Sep. 17, 2012, the entire contents of which are incorporated by reference herein.

### TECHNICAL FIELD OF THE INVENTION

The present invention concerns in vivo imaging and in particular a novel labelled compound suitable for in vivo imaging. Also provided by the present invention is a method for the preparation of the labelled compound of the invention, and a precursor compound useful in said method. The labelled compound of the invention is useful in the diagnosis of pathological conditions which comprise fibrosis.

### DESCRIPTION OF RELATED ART

Fibrosis is triggered as a response to tissue damage resulting from inflammation, infection or injury and forms part of all repair processes in tissue. In the case of on-going inflammation, infection and repeated injury, fibrosis scar tissue builds up and does not replace functional cells, which leads to abnormal organ function and eventually organ failure.

The clinical manifestations of fibrosis vary widely. Fibrosis is one of the major, classic pathological processes in medicine. It is a key component of multiple diseases that affect millions of people worldwide including:
a) Lung diseases such as idiopathic pulmonary fibrosis (lung fibrosis of unknown origin), asthma and chronic obstructive pulmonary disease
b) Scleroderma: a heterogeneous and life threatening disease characterised by the excessive extracellular matrix deposition within connective tissue of the body (i.e. skin and visceral organs)
c) Post-surgical scarring following transplantation
d) Diabetic retinopathy and age-related macular degeneration (fibrotic diseases of the eye and leading causes of blindness)
e) Cardiovascular disease including atherosclerosis and vulnerable plaque.
f) Kidney fibrosis linked to diabetes— diabetic nephropathy and glomerulosclerosis
g) IgA nephropathy (causes of kidney failure and the need for dialysis and retransplant)
h) Cirrhosis and biliary atresia (leading causes of liver fibrosis and failure)
i) Rheumatoid arthritis
j) Autoimmune diseases such as dermatomyositis
k) Congestive heart failure

Taking the example of cirrhosis, the clinical manifestations vary from no symptoms at all, to liver failure, and are determined by both the nature and severity of the underlying liver disease as well as the extent of hepatic fibrosis (reviewed by Zhou & Lu 2009 J Digestive Diseases; 10: 7-14). The common causes of liver fibrosis and cirrhosis include immune mediated damage, genetic abnormalities, and non-alcoholic steatohepatitis (NASH), which is particularly associated with diabetes and metabolic syndrome. There is a high incidence of metabolic syndrome in the western population. This syndrome typically occurs in individuals who are obese, have hyperlipidaemia and hypertension, and often leads to the development of type II diabetes. The hepatic manifestation of metabolic syndrome is non-alcoholic fatty liver disease (NAFLD), with an estimated prevalence in the USA of 24% of the population. A fatty liver represents the less severe end of a spectrum of NAFLD that may progress to NASH and ultimately to cirrhosis of the liver. The development of fibrosis demonstrates a risk of such progression, and is presently assessed by means of a liver biopsy. However, liver biopsy causes significant discomfort, is not without risk, is costly and suffers from sampling variability and inconsistent interpretation (Vuppalanchi & Chalasani 2009 Hepatology; 49(1): 306-317).

Fibroblast activation protein (FAP, also known as seprase) belongs to the prolyl peptidase family, which comprises serine proteases that cleave bioactive peptidase preferentially after proline residues. The prolyl peptidase family includes enzymes such as dipeptidase-IV (DPP-IV), DPP-II, DPP7, DPP8, and DPP9 and this family has been implicated in several diseases. FAP is a homodimer transmembrane serine protease which is selectively and highly expressed on activated fibroblasts. FAP is also a marker of tumour-associated fibroblasts. FAP expression precedes that of other fibrosis markers such as α-SMA.

Previously several N- and αC-substituted Gly-boro-Pro derivatives have been developed as FAP inhibitors. Radiolabeled derivatives of Gly-boro-pro have been reported by Zimmerman et at (WO 2010/036814). Zimmerman et at disclose the synthesis of novel ^{99m}Tc- and/or Re-labelled complexes of proline boronic acids. Compounds 1014, 1018 and 1020 of Zimmerman et at are reported to have IC₅₀ values of 21, 20 and 4 nM, respectively:

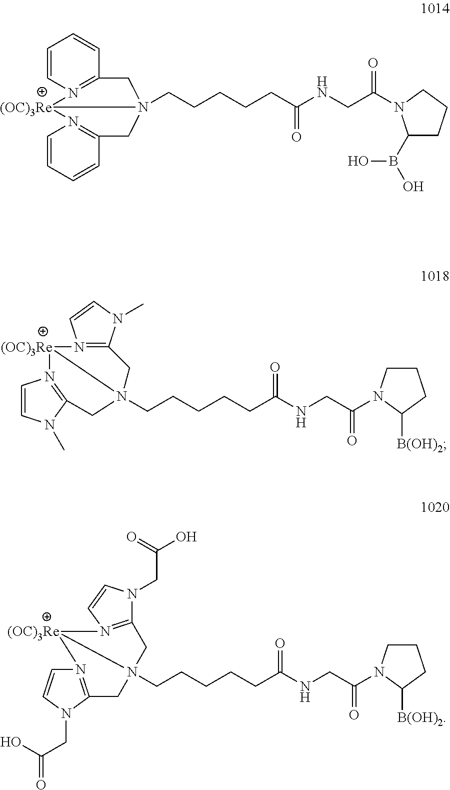

Ideally in addition to low nanomolar affinity a radiolabelled FAP inhibitor should at least 4-fold selectivity for FAP over DPP-IV. No data on selectivity of the above compounds is presented by Zimmerman et al. These compounds are described as useful for use in SPECT imaging, particularly in the radioimaging and radiotherapy of diseases characterised by overexpression of FAP and in particular cancer. However, biodistribution data presented by Zimmerman et at for ^{99m}Tc- and rhenium-labelled compounds (FIGS. 6 and 7 of Zimmerman et al) shows that the majority of the activity is either in the liver or small or large intestines, which in the context of in vivo imaging and in particular for detection of lesions in the liver is undesirable. The present inventors tried to obtain compound 1020 for comparative purposes but found that the synthetic description in Zimmerman et at lacked sufficient detail.

There is therefore a need for improved methods useful in the diagnosis of conditions comprising fibrosis, and for improved boronic acid compounds labelled via a chelate that are suitable for in vivo imaging.

### SUMMARY OF THE INVENTION

The present invention provides a compound having improved properties for use as an in vivo imaging agent as compared with known compounds. The binding properties, biodistribution and metabolic profile of the compound of the invention support its use as an in vivo diagnostic and imaging agent for fibrosis.

### DESCRIPTION OF THE PREFERRED EMBODIMENTS

In a first aspect, the present invention provides a compound of Formula I:

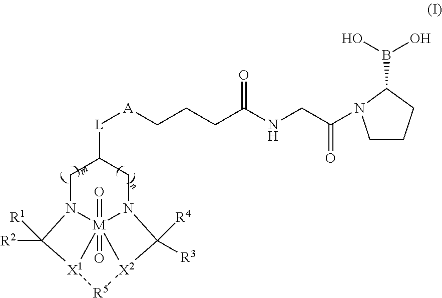

or a salt or solvate thereof;
wherein:
A is —(CH₂)ₒ—C(═O)—NH— or —(CH₂)ₚ—NH—C(═O)— wherein each of o and p is an integer between 0-4; ####
L is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl;
M is a metal ion selected from ^{99m}Tc, ¹⁸⁶Re and ¹⁸⁸Re; and,
either:
   X¹ and X² are both —CH₂—NH wherein each N is co-ordinated to M and R⁵ is not present; or,
   —X¹—R⁵—X²— is —C(CH₃)═N—O—H—O—N═C(CH₃)— wherein each N is co-ordinated to M.

Suitable salts according to the term “salt or solvate thereof” include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycollic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine. Suitable solvates according to the term “salt or solvate thereof” include those formed with ethanol, water, saline, physiological buffer and glycol.

The term “bivalent” (also commonly referred to as “divalent”) refers to an ion or molecule having a valence of two and can form two bonds with other ions or molecules.

The term “amino acid residue” refers to meant a residue of an L- or a D-amino acid, amino acid analogue (e.g. naphthylalanine) or amino acid mimetic which may be naturally occurring or of purely synthetic origin, and may be optically pure, i.e. a single enantiomer and hence chiral, or a mixture of enantiomers. Preferably the amino acids of the present invention are optically pure.

The term “carbohydrate residue” refers to an aldehyde or a ketone derivative of a polyhydric alcohol. It may be a monomer (monosaccharide), such as fructose or glucose, or two sugars joined together to form a disaccharide. Disaccharides include sugars such as sucrose, which is made of glucose and fructose. The term “sugar” includes both substituted and non-substituted sugars, and derivatives of sugars. Preferably, the sugar is selected from glucose, glucosamine, galactose, galactosamine, mannose, lactose, fucose and derivatives thereof, such as sialic acid, a derivative of glucosamine. The sugar is preferably α or β. The sugar may especially be a manno- or galactose pyranoside. The hydroxyl groups on the sugar may be protected with, for example, one or more acetyl groups. The sugar moiety is preferably N-acetylated. Preferred examples of such sugars include N-acetyl galactosamine, sialic acid, neuraminic acid, N-acetyl galactose, and N-acetyl glucosamine.

The term “alkyl” means straight-chain or branched-chain alkyl radical containing preferably from 1 to 4 carbon atoms. Examples of such radicals include methyl, ethyl, and propyl.

The term “co-ordinated” (also referred to as “complexed”) in the context of the present invention refers to the process where one or more atoms donate a pair of electrons to form a coordinate covalent bond to a metal ion.

A compound of Formula I is prepared by reaction of a suitable source of said metal ion with a precursor compound of Formula II. The precursor compound of Formula II forms a second aspect of the invention and is described in more detail below. The method to prepare the compound of the invention forms a third aspect of the present invention and is described in more detail below.

In one embodiment A is preferably —(CH₂)ₒ—C(═O)—NH— wherein o is an integer between 1-3, most preferably 2.

In another embodiment A is —(CH₂)ₚ—NH—C(═O)— wherein p is an integer between 1-3, most preferably 2.

In one embodiment m and n are both 1.

In another embodiment m and n are both 2.

In one embodiment R¹⁻⁴ are all hydrogen.

In another embodiment R¹⁻⁴ are all methyl.

In one embodiment M is ^{99m}Tc.

In another embodiment M is ¹⁸⁶Re.

In a further embodiment M is ¹⁸⁸Re.

In one embodiment X¹ and X² are both —CH₂—NH wherein each N is co-ordinated to M and R⁵ is not present.

In another embodiment —X¹—R⁵—X²— is —C(CH₃)═N—O—H—O—N═C(CH₃)— wherein each N is co-ordinated to M.

The bivalent linker group L preferably has 1-30 bivalent linker units, most preferably 1-20 linker units, and especially preferably 1-10 bivalent linker units. R′ as defined for the bivalent linker unit is preferably hydrogen. Preferred bivalent linker units are selected from —CH₂—, —(O—CH₂—CH₂)—, —C(═O)—NH—, —(O—CH₂—CH₂)—, and —CH₂—NH—. In one embodiment the bivalent linker group L is —CH₂—.

In a first preferred embodiment:
A is —(CH₂)₂—C(═O)—NH—;
L is —CH₂—
m and n are both 1; and,
X¹ and X² are both —CH₂—NH wherein each N is co-ordinated to M and R⁵ is not present.

In a second preferred embodiment:
A is —(CH₂)₂—NH—C(═O)—;
L is —CH₂—
m and n are both 2; and,
—X¹—R⁵—X²— is —C(CH₃)═N—O—H—O—N═C(CH₃)— wherein each N is co-ordinated to M.

Examples of preferred compounds of the present invention are rhenium-labelled (R)-(1-(2-(6-(4-((2-aminoethyl)amino)-3-(((2-aminoethyl)amino)methyl)butanamido)hexanamido)acetyl)pyrrolidin-2-yl)boronic acid, (Compound 1) and ^{99m}Tc-labelled (R)-(1-(2-(6-(4-((2-aminoethyl)amino)-3-(((2-aminoethyl)amino)methyl)butanamido)hexanamido)acetyl)pyrrolidin-2-yl)boronic acid (Compound 2). Synthesis and testing of these compounds is described in the examples below. The rhenium-labelled Compound 1 was tested in an in vitro assay and found to have high and selective affinity for FAP. The ⁹⁹mTc-labelled Compound 2 was demonstrated to have good biodistribution for in vivo imaging purposes as well as a good in vivo metabolic profile. These compounds compare favourably with the rhenium- and ^{99m}Tc-labelled compounds of the prior art.

In a second aspect, the present invention provides a precursor compound of Formula II:

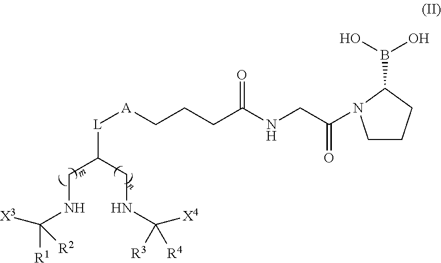

wherein:
A, L, m, n, and R¹⁻⁴ are as defined herein for Formula I;
X³ and X⁴ are either both —CH₂—NH₂ or both —C(CH₃)═N—OH.

In a first preferred embodiment of the precursor compound of the invention, X³ and X⁴ are both —CH₂—NH₂. For this preferred embodiment of the precursor compound of the invention, the preferences described previously for the first preferred embodiment of the compound of the invention for any feature in common between the two aspects also apply.

In a second preferred embodiment of the precursor compound of the invention, X³ and X⁴ are both —C(CH₃)═N—OH. For this preferred embodiment of the precursor compound of the invention, the preferences described previously for the second preferred embodiment of the compound of the invention for any feature in common between the two aspects also apply.

The precursor compounds of Formula II of the invention are obtained by linking an optionally protected carboxylic acid derivative 1 of the chelate linker moiety of Formula II with a glycine-boronoproline intermediate 2 as illustrated in Scheme 1 below:

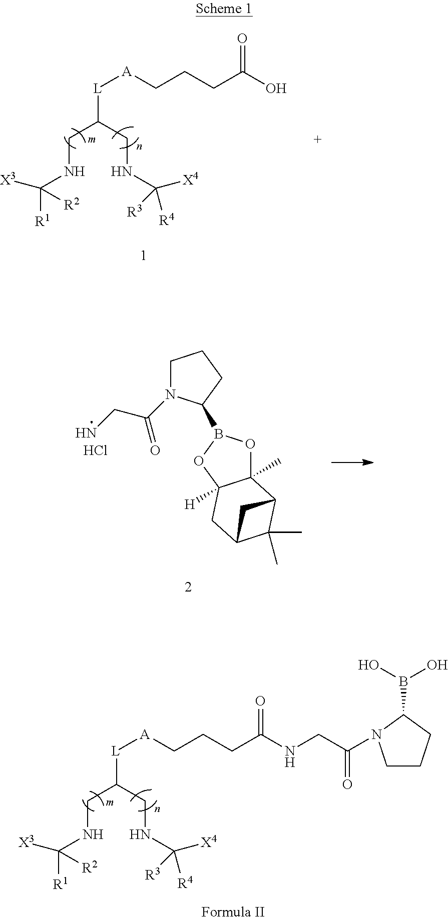

The groups L, A, m, n, R¹⁻⁴, X³ and X⁴ are as defined herein for Formula II. Where intermediate 1 is referred to as “protected” this refers to the inclusion of suitable protecting groups for any reactive groups other than the carboxylic acid, in order to avoid unwanted side reactions. In particular protecting groups may be included to protect any amine groups in 1.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well-known to those skilled in the art. Suitable protecting groups for amines include Boc (where Boc is tert-butyloxycarbonyl), Fmoc (where Fmoc is fluorenylmethoxycarbonyl), trifluoroacetyl, allyloxycarbonyl, Dde (1-(4,4-dimethyl-2,6-dioxocyclohexylidene)ethyl) or Npys (3-nitro-2-pyridine sulfenyl); and for carboxyl groups: methyl ester, tert-butyl ester or benzyl ester. Further information about protecting groups can be found in ‘Protective Groups in Organic Synthesis’, Theodora W. Greene and Peter G. M. Wuts, (Fourth Edition, John Wiley & Sons, 2006).

The glycine boronoproline intermediate 2 may be obtained by following the method described in Example 1 of Zimmerman et at (WO 2010/036814), which follows the literature procedure of Coutts et al (1996 J Med Chem; 39: 2087) as illustrated in Scheme 2:

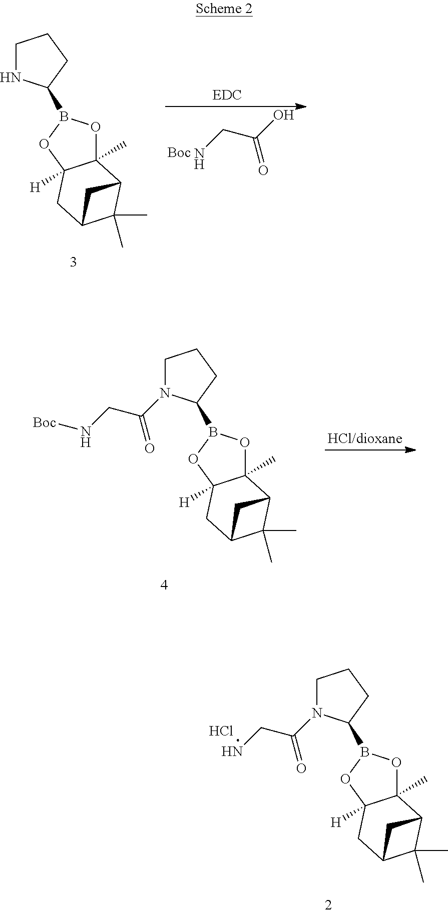

The chelate moiety (i.e. that part resulting from intermediate 1 of Scheme 1) of the compound of Formula II is a tetradentate ligand which is particularly suitable for the co-ordination of Tc and Re ions. Four donor atoms are arranged such that a 5- or 6-membered chelate ring results (by having a non-coordinating backbone of either carbon atoms or non-coordinating heteroatoms linking the metal donor atoms). The metal complex formed between the chelate moiety and the metal ion is “resistant to transchelation”, i.e. does not readily undergo ligand exchange with other potentially competing ligands for the metal coordination sites. Potentially competing ligands may be in the precursor compound itself, or in other excipients in the preparation in vitro (e.g. radioprotectants or antimicrobial preservatives used in the preparation), or endogenous compounds in vivo (e.g. glutathione, transferrin or plasma proteins). A preferred chelate moiety for inclusion in the precursor compound of the present invention is either an N₄ ligand (an open chain or macrocyclic ligands having a tetraamine, amidetriamine or diamidediamine donor set) or a diamine dioxime ligand. Jurisson et at (1999 Chem Rev; 99: 2205-2218) describes these ligand systems in more detail.

One particularly preferred chelate moiety for inclusion in the precursor compound of the present invention is the tetraamine ligand system disclosed in WO 2006/008496. Example 1 of WO 2006/008496 describes the synthesis of the following carboxylic acid derivative:

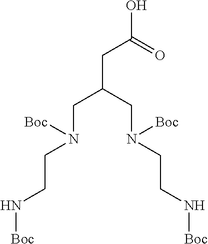 [SMILES: CC(C)(C)OC(=O)NCCN(CC(CC(=O)O)CN(CCNC(=O)OC(C)(C)C)C(=O)OC(C)(C)C)C(=O)OC(C)(C)C]

The synthesis of the above-illustrated compound can be readily adapted by means known to those of skill in the art in order to obtain other intermediates 1 as illustrated in Scheme 1 above.

Another particularly preferred chelate moiety for inclusion in the precursor compound of the present invention is the diamine dioxime ligand system disclosed in WO 2003/006070. Example 6 of WO 2003/006070 describes the synthesis of a carboxylic acid derivative of a diamine dioxime ligand system that could be used as an intermediate 1 as illustrated in Scheme 1 above.

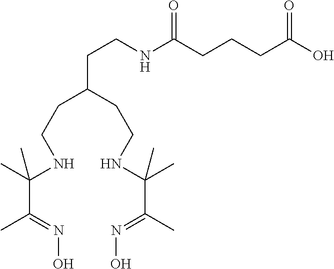 [SMILES: C/C(=N\O)C(C)(C)NCCC(CCNC(=O)CCCC(=O)O)CCNC(C)(C)/C(C)=N/O]

The synthesis of the above-illustrated compound can be readily adapted by means known to those of skill in the art in order to obtain other intermediates 1.

In a third aspect, the present invention provides a method for the preparation of the compound of Formula I as defined hereinabove for the first aspect of the invention wherein said method comprises reaction of the precursor compound of second aspect of the invention with a suitable source of said metal ion M as defined hereinabove for the first aspect of the invention. Any preferred embodiments set out for features of the first and second aspects of the invention equally apply for this third aspect of the invention in respect of equivalent features.

The “suitable source” of said metal ion is commonly the pertechnetate ion (TcO₄⁻) when the metal ion is technetium, and the perrhennate ion (ReO₄⁻) when the metal ion is rhenium, both of which feature the respective metal ion in the +7 oxidation state. Neither pertechnetate nor perrhenate readily forms a metal complex and therefore the preparation of these metal complexes requires the addition of a suitable reducing agent such as stannous ion to facilitate co-ordination by reducing the oxidation state of the metal ion to the lower oxidation states, usually +1 to +5. Rhenium is harder to reduce than technetium and requires harsher reaction conditions and longer reaction times than for technetium. The solvent used may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline. The reader is referred to “Metal-based Radiopharmaceuticals” by Roger Alberto (Chapter 9 of “Bioinorganic Medicinal Chemistry” 2011 Wiley-VCH; Enzo Alessio, Ed.) for a more detail on methods of labelling with ^{99m}Tc, ¹⁸⁶Re and ¹⁸⁸Re.

In a fourth aspect, the present invention provides a pharmaceutical composition comprising the compound of the first aspect of the invention together with a biocompatible carrier suitable for mammalian administration.

The “biocompatible carrier” is a fluid, especially a liquid, in which the compound is suspended or dissolved, such that the composition is “suitable for mammalian administration”, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The pharmaceutical composition of the invention is suitably supplied in a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose (or “unit dose”) and are therefore preferably a disposable or other syringe suitable for clinical use. The pre-filled syringe is suitably provided with a syringe shield to protect the operator from radioactive dose. Suitable such radiopharmaceutical syringe shields are known in the art and preferably comprise either lead or tungsten.

The pharmaceutical composition of the present invention may be prepared from a kit. Alternatively, the pharmaceutical composition may be prepared under aseptic manufacture conditions to give the desired sterile product. The pharmaceutical composition may also be prepared under non-sterile conditions, followed by terminal sterilisation using e.g. gamma-irradiation, autoclaving, dry heat or chemical treatment (e.g. with ethylene oxide). Preferably, the pharmaceutical composition of the present invention is prepared from a kit.

In a fifth aspect, the present invention provides such a kit for carrying out the method of the third aspect of the invention wherein said kit comprises the precursor compound of the second aspect of the invention. The precursor compound is preferably provided in sterile non-pyrogenic form, so that reaction with a sterile source of the metal ion M as defined for the first aspect of the invention gives the desired pharmaceutical composition with the minimum number of manipulations. Such considerations are particularly important for ease of handling and hence reduced radiation dose for the radiopharmacist. Hence, the reaction medium for reconstitution of such kits is preferably a biocompatible carrier as defined above, and is most preferably aqueous. The precursor compounds for use in the kit may be employed under aseptic manufacture conditions to give the desired sterile, non-pyrogenic material. The precursor compounds may also be employed under non-sterile conditions, followed by terminal sterilisation using as described above. Preferably, the precursor compounds are employed in sterile, non-pyrogenic form.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. Suitable radioprotectants are chosen from: ascorbic acid, para-aminobenzoic acid (i.e. 4-aminobenzoic acid), gentisic acid (i.e. 2,5-dihydroxybenzoic acid) and salts thereof with a biocompatible cation.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the imaging agent product itself. The antimicrobial preservative may, however, also optionally be used to inhibit the growth of potentially harmful micro-organisms in one or more components of the kit prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS (i.e. tris(hydroxymethyl)aminomethane), and pharmaceutically acceptable bases such as sodium carbonate, sodium bicarbonate or mixtures thereof.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

In a sixth aspect, the present invention provides an in vivo imaging method comprising:
(i) administering to a subject the compound of the first aspect of the invention;
(ii) allowing said compound to bind to a biological target in said subject;
(iii) detecting by an in vivo imaging procedure signals emitted by the metal ion of said compound;
(iv) generating an image representative of the location and/or amount of said signals.

The “subject” can be any human or animal subject. Preferably the subject is a mammal. Most preferably, said subject is an intact mammalian body in vivo. In an especially preferred embodiment, the subject of the invention is a human.

The step of “administering” the compound is preferably carried out parenterally, and most preferably intravenously. The intravenous route represents the most efficient way to deliver the compound throughout the body of the subject and into contact with FAP-expressing tissue in said subject. The compound of the invention is preferably administered as the pharmaceutical composition of the invention, as defined hereinabove. Preferably, the compound of the invention for use in the in vivo imaging method of the invention is labelled with ^{99m}Tc.

The in vivo imaging method of the invention can also be understood to begin from an alternative step (i) wherein said subject is provided with the compound of the invention has been previously administered.

Following the administering step and preceding the detecting step, the compound is allowed to bind to a biological target in said subject. Suitably, said biological target is FAP. The compound moves dynamically through the subject's body, coming into contact with various tissues therein. Once the compound comes into contact with FAP, a specific interaction takes place such that clearance of the compound from tissue with FAP takes longer than from tissue without, or expressing less FAP. A certain point in time is reached when detection of compound specifically bound to FAP is enabled as a result of the ratio between compound bound to tissue with FAP versus that bound in tissue expressing less (or no) FAP.

The step of “detecting signals” involves detection of gamma rays emitted by ^{99m}Tc by means of a single-photon emission computed tomography (SPECT) camera.

The step of “generating an image” is carried out by a computer which applies a reconstruction algorithm to the acquired signal data to yield a dataset. This dataset is then manipulated to generate an image showing the location and/or amount of signals emitted by the ^{99m}Tc.

For equivalent features, the preferred aspects as set out for other aspects of the invention are equally applicable for the sixth aspect of the invention.

In a preferred embodiment, the in vivo imaging method of the invention comprises the subsequent step (v) of determining the distribution and extent of FAP expression in said subject wherein said expression is directly correlated with said signals.

In a further preferred embodiment, the in vivo imaging method of the invention is carried out repeatedly during the course of a treatment regimen for said subject. In this way, the progress of treatment can be monitored and decisions on the most appropriate treatment for said subject can be facilitated.

In a seventh aspect, the present invention provides a method for the diagnosis of a condition in which FAP is upregulated wherein said method comprises the in vivo imaging method of the invention comprising steps (i)-(v) together with the further subsequent step (vi) of attributing the distribution and extent of FAP expression to a particular clinical condition, referred to hereunder as an FAP condition.

An “FAP condition” refers to a pathological condition characterised by abnormal expression of FAP and typically over-expression of FAP. Examples of such conditions where the in vivo imaging method of the invention finds use include any condition that comprises fibrosis. Given that FAP expression precedes that of other fibrosis markers the in vivo imaging method of the invention is particularly suitable in the diagnosis of the early stages of fibrosis. Examples of FAP conditions include lung diseases such as idiopathic pulmonary fibrosis (lung fibrosis of unknown origin), asthma and chronic obstructive pulmonary disease, scleroderma: a heterogeneous and life threatening disease characterised by the excessive extracellular matrix deposition within connective tissue of the body (i.e. skin and visceral organs), post-surgical scarring following transplantation, diabetic retinopathy and age-related macular degeneration (fibrotic diseases of the eye and leading causes of blindness), cardiovascular disease including atherosclerosis and vulnerable plaque, kidney fibrosis linked to diabetes—diabetic nephropathy and glomerulosclerosis, IgA nephropathy (causes of kidney failure and the need for dialysis and retransplant), cirrhosis and biliary atresia (leading causes of liver fibrosis and failure), rheumatoid arthritis, autoimmune diseases such as dermatomyositis and congestive heart failure.

Preferably, for the method for the diagnosis of a condition in which FAP is upregulated, said condition is liver fibrosis, atherosclerosis, vulnerable plaque or congestive heart failure.

The present invention also provides the compound of the invention for use in either the in vivo imaging method of the sixth aspect of the invention or the method of diagnosis of the seventh aspect of the invention, wherein the broad and suitable definitions for these aspects equally apply here.

Furthermore, the present invention also provides for use of the compound of the first aspect of the invention in the manufacture of an in vivo imaging agent for use in either the in vivo imaging method of the sixth aspect of the invention or the method of diagnosis of the seventh aspect of the invention, wherein the broad and suitable definitions for these aspects equally apply here. The in vivo imaging agent in this aspect of the invention is preferably the pharmaceutical composition of the fourth aspect of present invention as defined hereinabove.

The invention is described in more detail in the following non-limiting examples.

### BRIEF DESCRIPTION OF THE EXAMPLES

Example 1 describes the synthesis of a compound of the invention, (R)-(1-(2-(6-(4-((2-aminoethyl)amino)-3-(((2-aminoethyl)amino)methyl)butanamido)hexanamido)acetyl)pyrrolidin-2-yl)boronic acid, dioxorhenium(V) chelate (Compound 1).

Example 2 describes the in vitro screening of Compound 1.

Example 3 describes the ^{99m}Tc labelling of (R)-(1-(2-(6-(4-((2-aminoethyl)amino)-3-(((2-aminoethyl)amino)methyl)butanamido)hexanamido)acetyl)pyrrolidin-2-yl)boronic acid (Compound 2).

Example 4 describes the biodistribution of Compound 2 in naive rats.

Example 5 describes the metabolism study of Compound 2 in naive rats.

### List of Abbreviations Used in the Examples

AMC=Aminomethylcoumarin

Boc=tert-butoxycarbonyl

DCM=Dichloromethane

DMSO=dimethyl sulfoxide

DPP-IV=dipeptidyl peptidase IV

EDC=1-Ethyl-3-(3-dimethylaminopropyl)carbodiimide

FAP=fibroblast activation protein

HOBt=Hydroxybenzotriazole

KHSO₄=Potassium hydrogen sulphate

MgSO₄=Magnesium sulphate

N₂=Nitrogen (gas)

Na₂CO₃=Sodium carbonate

### EXAMPLES

### Example 1

### Synthesis of (R) (1-(2-(6-(4-((2-aminoethyl)amino)-3-(((2-aminoethyl)amino)methyl)butanamido)hexanamido)acetyl)pyrrolidin-2-yl)boronic acid, dioxorhenium(V) chelate (Compound 1)

The synthesis is based in part on the disclosures of Coutts et at (1996 J Med Chem; 39: 2087-2094), Gibson et at (2002 Organic Process Research & Development; 6: 814-816) and Kelly et at (1993 Tetrahedron; 49(5):1009-1016).

### Di-tert-butyl ((2,2,12,12-tetramethyl-4,10-dioxo-7-(2-oxo-2-((6-oxo-6-((2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)amino)hexyl)amino)ethyl)-3,11-dioxa-5,9-diazatridecane-5,9-diyl)bis(ethane-2,1-diyl))dicarbamate

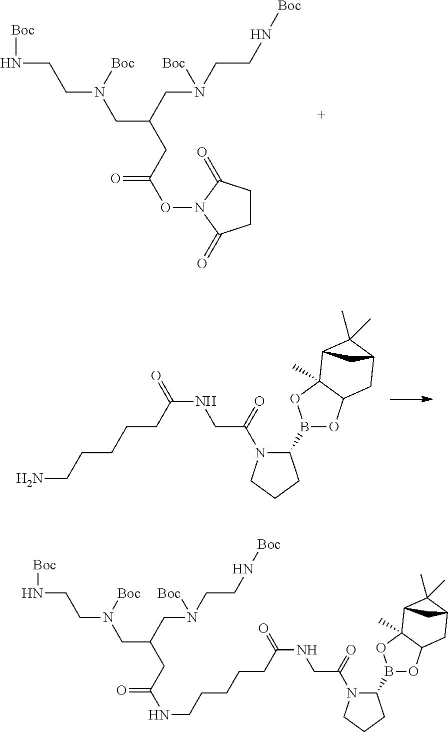 [SMILES: CC(C)(C)OC(=O)NCCN(CC(CC(=O)ON1C(=O)CCC1=O)CN(CCNC(=O)OC(C)(C)C)C(=O)OC(C)(C)C)C(=O)OC(C)(C)C ; CC1(C)[C@@H]2CC3OB([C@@H]4CCCN4C(=O)CNC(=O)CCCCCN)O[C@@]3(C)[C@H]1C2 ; CC(C)(C)OC(=O)NCCN(CC(CC(=O)NCCCCCC(=O)NCC(=O)N1CCC[C@H]1B1OC2C[C@@H]3C[C@@H](C3(C)C)[C@]2(C)O1)CN(CCNC(=O)OC(C)(C)C)C(=O)OC(C)(C)C)C(=O)OC(C)(C)C]

2,5-dioxopyrrolidin-1-yl 4-((tert-butoxycarbonyl)(2-((tert-butoxycarbonyl)amino)ethyl)amino)-3-(((tert-butoxycarbonyl)(2-((tert-butoxycarbonyl)amino)ethyl)amino)methyl)butanoate (90 mg, 0.13 mmol), 6-amino-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide (58 mg, 0.13 mol) were added to DCM (10 ml). 4-Methyl morpholine was added and the resulting solution was left stirring for 1 hour under Argon. The reaction solution was washed with KHSO₄ (1M, 10 ml), Water (10 ml) and Na₂CO₃ (1M, 10 ml) before drying with MgSO₄, filtered and concentrated to dryness to give the title compound as a white foam (103 mg, 0.1 mmol, 80%).

The material was used in subsequent step without any further purification.

### 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide hydrochloride

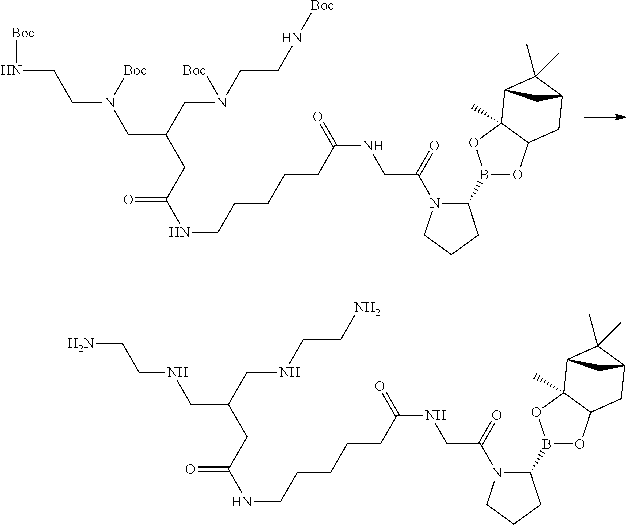 [SMILES: CC(C)(C)OC(=O)NCCN(CC(CC(=O)NCCCCCC(=O)NCC(=O)N1CCC[C@H]1B1OC2C[C@@H]3C[C@@H](C3(C)C)[C@]2(C)O1)CN(CCNC(=O)OC(C)(C)C)C(=O)OC(C)(C)C)C(=O)OC(C)(C)C ; CC1(C)[C@@H]2CC3OB([C@@H]4CCCN4C(=O)CNC(=O)CCCCCNC(=O)CC(CNCCN)CNCCN)O[C@@]3(C)[C@H]1C2]

Di-tert-butyl ((2,2,12,12-tetramethyl-4,10-dioxo-7-(2-oxo-2-((6-oxo-6-((2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)amino)hexyl)amino)ethyl)-3,11-dioxa-5,9-diazatridecane-5,9-diyl)bis(ethane-2,1-diyl))dicarbamate (200 mg, 0.19 mmol) was dissolved in diethyl ether (2 ml) and added HCl in diethyl ether (2M, 4.8 ml, 8.0 mmol) resulting in instant precipitation. The resulting suspension was left stirring for 1 hour before concentrated to dryness to give crude 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide tetra hydrochloride (150 mg).

The material was used in subsequent step without any further purification.

### 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide

6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide, tetra hydrochloride (40 mg, 0.058 mmol) was dissolved in diethyl ether (5 ml) and CH₂Cl₂ (2 ml). 1,1,3,3-Tetramethylguanidine (7.25 μl, 0.058 mmol) was added with added resulting in instant fogging. The suspension was stirred until the suspension was homogeneous and milky.

The reaction suspension was filtered and concentrated to dryness. The resulting yellow oil was re-dissolved in DCM to give a clear solution before again concentrated to dryness to give 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide as free base. The material was used in subsequent step without any further purification.

### 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide, dioxorhenium(V) salt

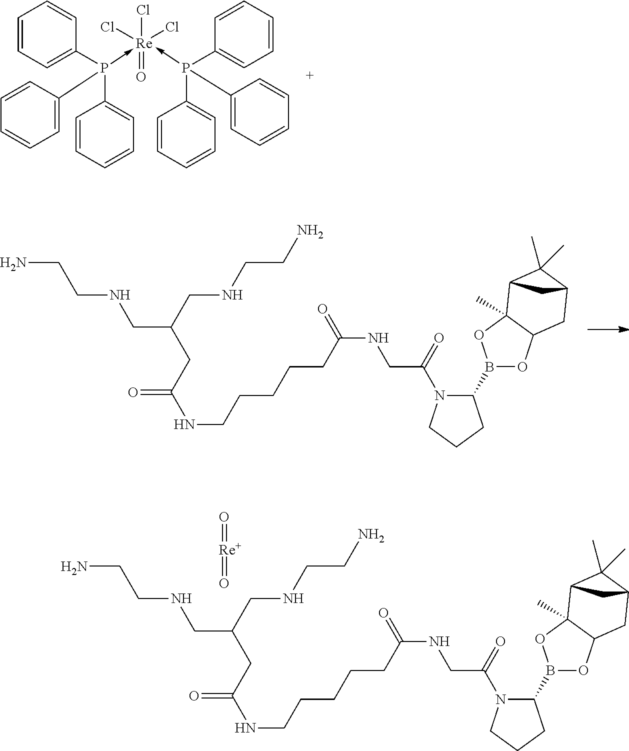

6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide (90 mg, 0.15 mmol) was dissolved in CH₂Cl₂ (2 ml) to give a clear solution. Trichlorooxobis(triphenylphosphine)rhenium(V) (121 mg, 0.15 mmol) was added to give a green suspension that dissolved over 2 min with a colour change from green to brown.

After 24 hours of stirring under Argon the reaction was concentrated to dryness to give a brown crude. The material was used in subsequent step without any further purification.

### (R)-(1-(2-(6-(4-((2-aminoethyl)amino)-3-(((2-aminoethyl)amino)methyl)butanamido)hexanamido)acetyl)pyrrolidin-2-yl)boronic acid, dioxorhenium(V)salt (Compound 1)

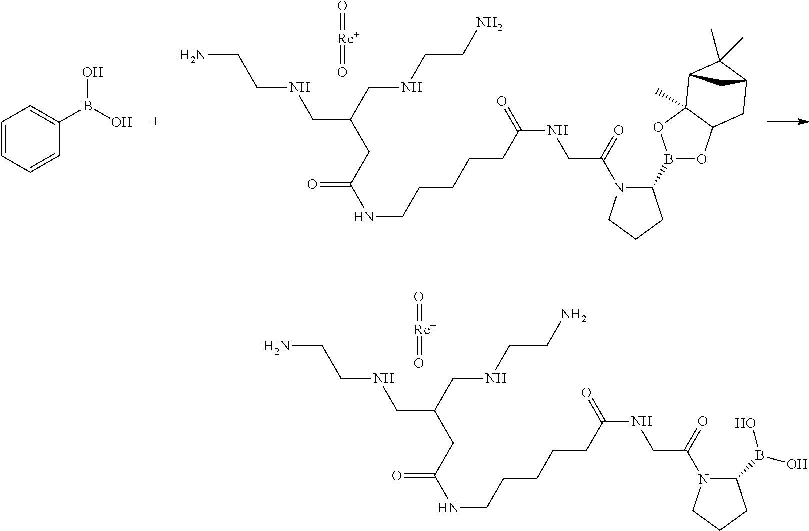

Phenylboronic acid (17.75 mg, 0.15 mmol), water (3 ml) and TBME were added to the crude 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide, dioxorhenium(V) salt (122 mg, 0.15 mmol) and the resulting 2 phase reaction suspension was left stirring at room temperature for 24 hours after which the phases were separated and the aqueous phase concentrated to dryness at low temperature to give a black oil.

The black crude was purified by preparative HPLC to give Compound 1 (6.2 mg) as a white solid after freeze-drying. For preparative HPLC a Waters Corporation LCT Premier—TOF mass spectrometer was used and a preparative HPLC system consisting of Beckman Gold Solvent delivery module 126 w. The column was a Phenomenex Luna (5m C18 (2) 250×21.20 mm). UV-VIS Detector model 166 and Fraction Collector, ISCO Foxy 2200 were used. The method used was: 5 to 40% B over 40 min, where A=water/0.1% TFA, B=ACN, flow: 10 mL/min, UV det. 214 nm.

Identity was confirmed by MS-TOF. Expected m/z [M⁺]=704.29 Found [M⁺−H₂O]=686.04, and [((M⁺−H2O)+H⁺)/2]=343.52

### Example 2

### In Vitro Screening

FAP and DPP-IV assay kits supplied by BPS Bioscience were used to determine the ability of Compound 1 and reference FAP and DPP-IV compounds to inhibit the enzymatic activity of the recombinant human FAP and DPP-IV enzymes.

NVP DPP 728 hydrochloride provided by Tocris Bioscience was used as the reference DPP-IV inhibitor. The known compound (R)-(1-(2-(1-naphthamido)acetyl)pyrrolidin-2-yl)boronic acid was used as the reference FAP inhibitor.

The assay uses the fluorogenic substrate Gly-Pro-Aminomethylcoumarin (AMC) to measure either FAP or DPP-IV activity. Cleavage of the peptide bond by either FAP or DPP-IV releases the free AMC group, resulting in fluorescence than can be analysed using an excitation wavelength of 350-380 nm (325 nm used) and an emission wavelength of 440-460 nm (450 nm used).

The enzyme activities were assayed in a total volume of 100 uL for 10 min (DPP-IV) and 30 min (FAP) at 22° C. The inhibitors were dissolved in DMSO. IC₅₀ values were computed using GraphPad Prism 4.

| | FAP | DPP-IV |
|---|---|---|
| Compound | IC50 (nM) | IC50 (nM) |
| DPP-IV reference | >100000 | 9 |
| FAP reference | 6.4 | >50000 |
| Compound 1 | 9 | >50000 |

### Example 3

### ^{99m}Tc-Radiolabeling of (R) (1-(2-(6-(4-((2-aminoethyl)amino)-3-(((2-aminoethyl)amino)methyl)butanamido)hexanamido)acetyl)pyrrolidin-2-yl)boronic acid (Compound 2)

### (R) (1-(2-(6-(4-((2-aminoethyl)amino)-3-(((2-aminoethyl)amino)methyl)butanamido)hexanamido)acetyl)pyrrolidin-2-yl)boronic acid (Compound 2)

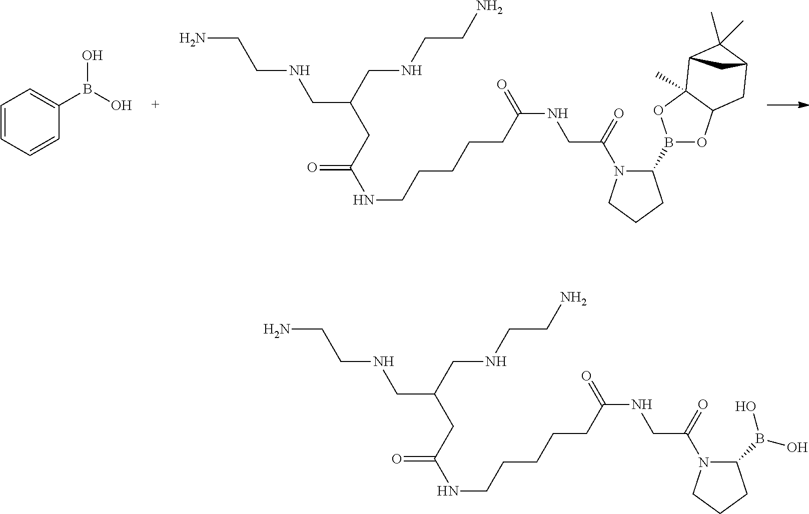 [SMILES: OB(O)c1ccccc1 ; CC1(C)[C@@H]2CC3OB([C@@H]4CCCN4C(=O)CNC(=O)CCCCCNC(=O)CC(CNCCN)CNCCN)O[C@@]3(C)[C@H]1C2 ; NCCNCC(CNCCN)CC(=O)NCCCCCC(=O)NCC(=O)N1CCC[C@H]1B(O)O]

Phenylboronic acid (6.8 mg, 0.06 mmol), water (3 mL) and DCM (3 mL) were added to the crude compound 6-(4-((2-aminoethyl)amino)-3-(((2-aminoethyl)amino)methyl)butanamido)-N-(2-oxo-2-((2R)-2-((3aS,4S,6S)-3a,5,5-trimethylhexahydro-4,6-methanobenzo[d][1,3,2]dioxaborol-2-yl)pyrrolidin-1-yl)ethyl)hexanamide hydrochloride (15 mg, 0.024 mmol) and the resulting 2 phase reaction suspension was left stirring at room temperature for 24 hours after which the phases were separated and the aqueous phase freeze-dried to give Compound 2. Material not further purified but used as crude. Identity was confirmed by MS-TOF. Expected m/z [M+H⁺]=486.37 Found [M+H⁺]=486.36

### ^{99m}Tc-compound 2

A lyophilised kit containing the following formulation (Vial 1) was prepared:

| Component | M. Wt | mg | μmoles |
|---|---|---|---|
| SnCl₂•2H₂O | 225.63 | 0.016 | 0.07 |
| MDP(H₄) | 176.00 | 0.025 | 0.14 |
| NaHCO₃ | 84.01 | 4.5 | 53.6 |
| Na₂CO₃ | 105.99 | 0.6 | 5.66 |
| NaPABA | 159.12 | 0.2 | 1.26 |

Vial 2 containing 100 μg Compound 2 in 100 μL methanol was added to vial 1. ^{99m}Tc-pertechnetate eluate from Drytec™ generator (GE Healthcare, 1 mL, 484 MBq) was then added to the vial, and the solution allowed to stand at room temperature for 20 min before HPLC purification. The RCP was >95%.

^{99m}Tc-Compound 2 was purified by HPLC(R_{T}=8.8 min) in 57% yield using a Phenomenex C18 (150×4.6 mm), 5 μm Luna column and 0.1% TFA in H₂O/acetontrile as mobile phase with a flow rate of 1 mL/minute.

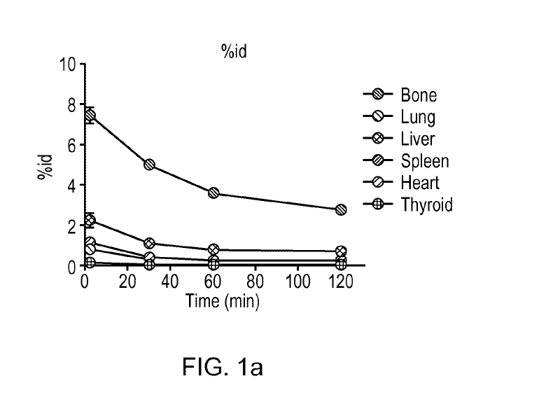

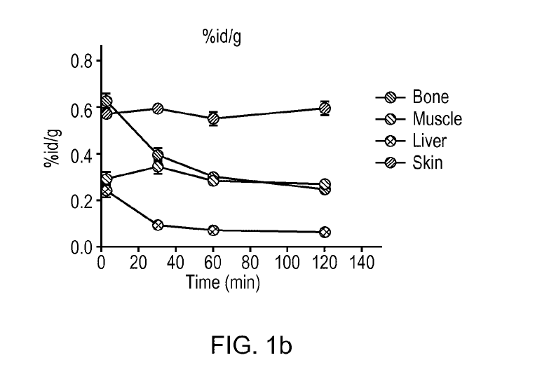

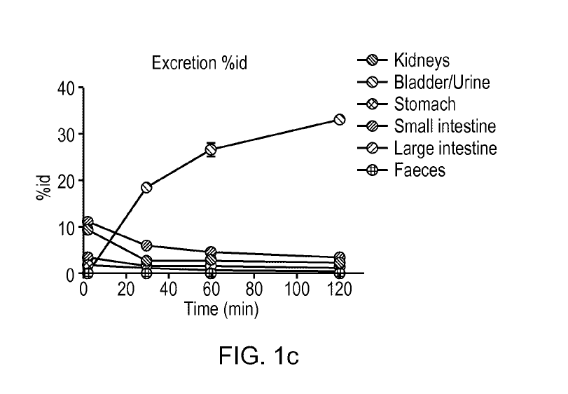

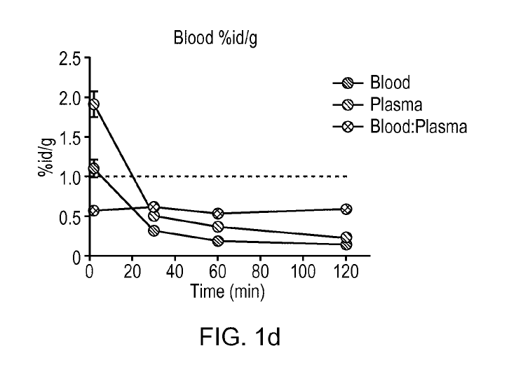

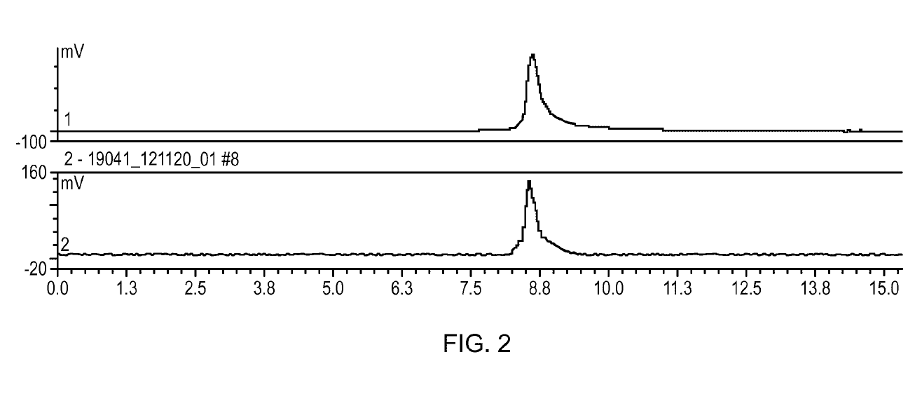

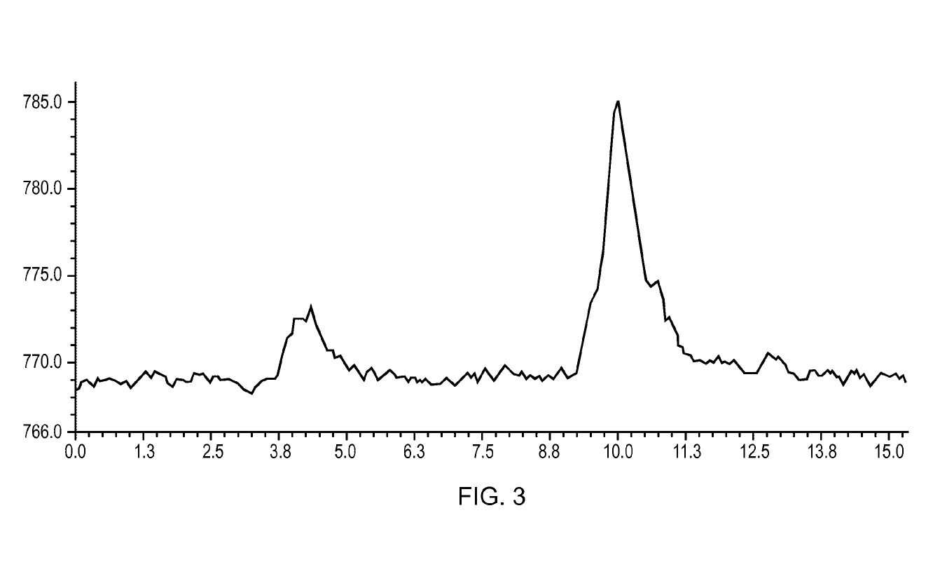

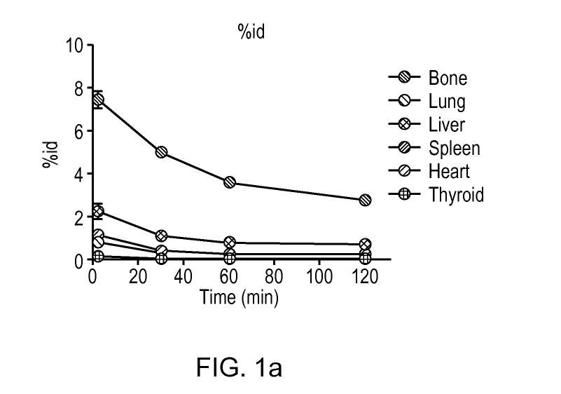

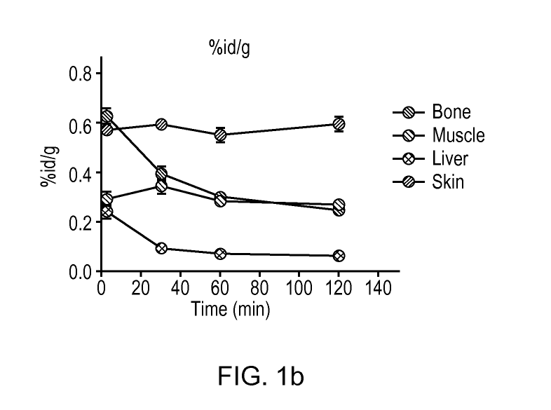

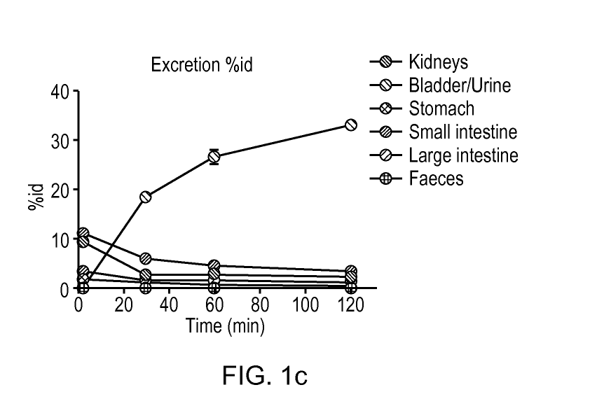

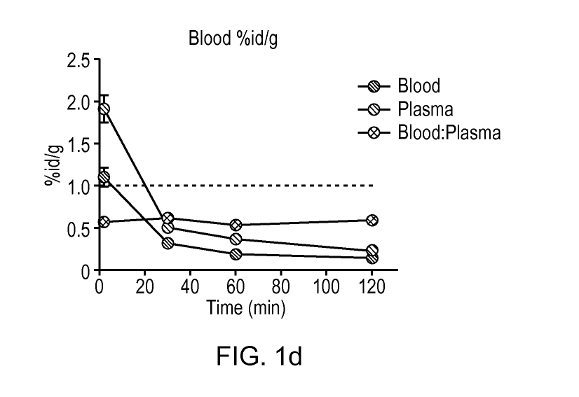

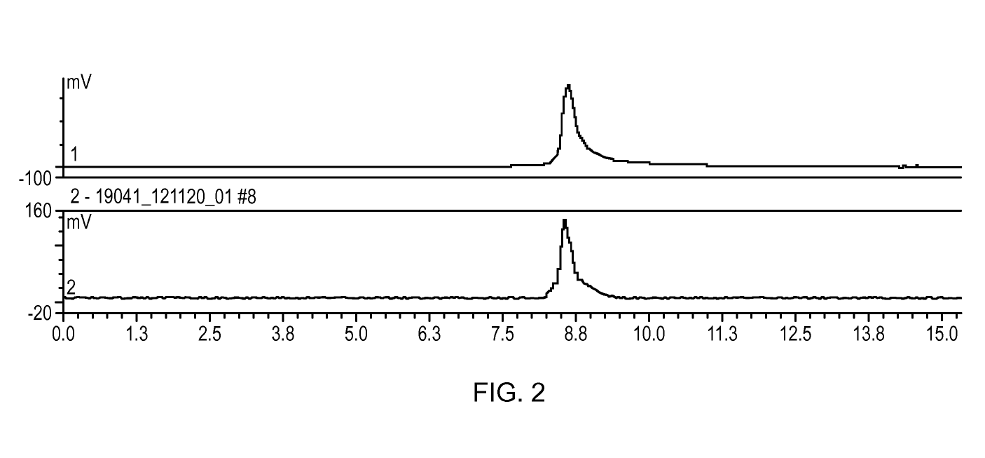

The ^{99m}Tc labeled Compound 2 was purified directly into 0.5 mL 50 mM phosphate buffer solution. After formulation in 10% ethanol/50 mM phosphate buffer solution at 20 MBq/mL, ^{99m}Tc-Compound 2 was found to be very stable over a period of 3 hr. FIG. 2 shows the Compound 2 reaction mixture (top trace) and the purified Compound 2 at 3 hours post-formulation (bottom trace).

### Example 4

### Biodistribution of ^{99m}Tc-Compound 2 in Naive Rats

Male Sprague-Dawley rats (231±10 g) were group housed in batches of four, with ad libitum access to food and water. Animals (n=12) were injected with ^{99m}Tc-Compound 2 (0.3 mL, 4-5MBq/animal), as an intravenous bolus via the tail vein. At various times post injection (2, 30, 60 and 120 minutes) animals were euthanised (n=3 per timepoint), dissected. Data are shown in the table below:

| | Time post injection (minutes) | | | |
|---|---|---|---|---|
| Tissue | 2 | 30 | 60 | 120 |
| Bone | 7.49 ± 0.79 | 5.03 ± 0.14 | 3.57 ± 0.08 | 2.82 ± 0.07 |
| Liver | 2.28 ± 0.34 | 1.06 ± 0.12 | 0.79 ± 0.02 | 0.63 ± 0.05 |
| Kidney | 9.32 ± 0.39 | 2.51 ± 0.27 | 2.49 ± 0.13 | 2.10 ± 0.33 |
| Blad- | 0.10 ± 0.01 | 18.56 ± 0.39 | 26.45 ± 1.97 | 33.11 ± 1.86 |
| der/ | | | | |
| Urine | | | | |
| Blood | 13.62 ± | 4.87 ± 0.54 | 2.88 ± 0.07 | 1.80 ± 0.03 |
| | 1.07 | | | |
| Plasma | 1.00 ± 0.14 | 0.39 ± 0.03 | 0.23 ± 0.01 | 0.13 ± 0.02 |

Data above are presented as % injected dose (mean±SD; n=3 animals per timepoint).

### Example 5

### Metabolism Study of Compound 2 in Naive Rats

The in vivo metabolic profile of Compound 2 was determined after intravenous administration to male rats. Post mortem blood was obtained at 60 minutes post injection and centrifuged to separate plasma.

Analysis was carried out by HPLC with prior extraction of Compound 2 from plasma using solid phase extraction (HLB cartridges; Waters). Briefly, cartridges were conditioned with 5 ml, acetonitrile, followed by 2×5 mL water. Plasma was loaded onto the cartridge and then washed with water (2% acetonitrile). Compound 2 was eluted from the cartridge with 7.5 mL water (0.1% TFA) with acetonitrile (0.1% TFA) as a 50:50 ratio.

The extract was evaporated to dryness and reconstituted in 2 mL starting mobile phase (4% acetonitrile (0.1% TFA) and water (0.1% TFA).

0.5 mL of the reconstituted extract anlaysed by HPLC using the same analytical conditions as mentioned above in Example 3 for Compound 2.

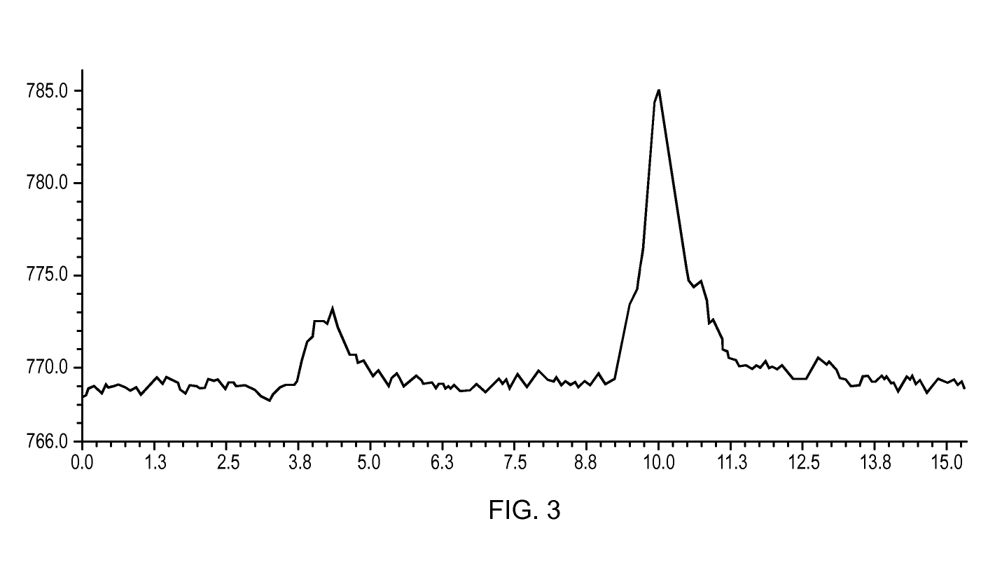

Approximately 80% of Compound 2 was still present in the plasma sample taken 60 minutes after injection of Compound 2 (see FIG. 3).

1. A compound of Formula I: 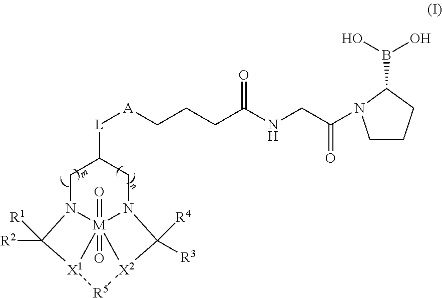
or a salt or solvate thereof;
wherein:
A is —(CH₂)ₒ—C(═O)—NH— or —(CH₂)ₚ—NH—C(═O)— wherein each of o and p is an integer between 0-4;
L is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl;
M is a metal ion selected from ^{99m}Tc, ¹⁸⁶Re and ¹⁸⁸Re; and,
either:
X¹ and X² are both —CH₂—NH wherein each N is co-ordinated to M and R⁵ is not present; or,
—X¹—R⁵—X²— is —C(CH₃)═N—O—H—O—N═C(CH₃)— wherein each N is co-ordinated to M.

2. The compound as defined in claim 1 wherein m and n are both 1.

3. The compound as defined in claim 1 wherein m and n are both 2.

4. The compound as defined in claim 1 wherein X¹ and X² are both —CH₂—NH₂ wherein each N is co-ordinated to M and R⁵ is not present.

5. The compound as defined in claim 1 wherein —X¹—R⁵—X²— is —C(CH₃)═N—O—H—O—N═C(CH₃)— wherein each N is co-ordinated to M.

6. The compound as defined in claim 1 wherein each of R¹⁻⁴ is hydrogen.

7. The compound as defined in claim 1 wherein each of R¹⁻⁴ is methyl.

8. The compound as defined in claim 1 wherein said metal ion is ^{99m}Tc.

9. A precursor compound of Formula II: 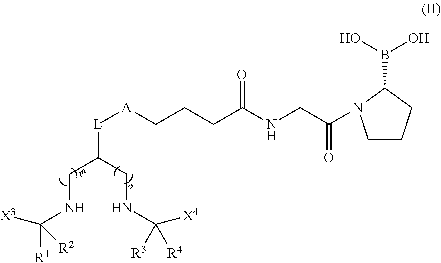
wherein:
A is —(CH₂)ₒ—C(═O)—NH— or —(CH₂)ₚ—NH—C(═O)— wherein each of o and p is an integer between 0-4;
L is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl; and,
X³ and X⁴ are either both —CH₂—NH₂ or both —C(CH₃)═N—OH.

10. The precursor compound as defined in claim 9 wherein X³ and X⁴ are both —CH₂—NH₂.

11. The precursor compound as defined in claim 9 wherein X³ and X⁴ are both —C(CH₃)═N—OH.

12. A method for the preparation of the compound of Formula I: 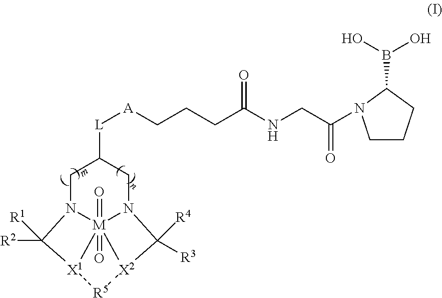
or a salt or solvate thereof;
wherein:
A is —(CH₂)ₒ—C(═O)—NH— or —(CH₂)ₚ—NH—C(═O)— wherein each of o and p is an integer between 0-4;
L is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl;
M is a metal ion selected from ^{99m}Tc, ¹⁸⁶Re and ¹⁸⁸Re; and,
either:
X¹ and X² are both —CH₂—NH wherein each N is co-ordinated to M and R⁵ is not present; or,
—X¹—R⁵—X²— is —C(CH₃)═N—O—H—O—N═C(CH₃)— wherein each N is co-ordinated to M,
wherein said method comprises reacting a precursor compound of Formula II: 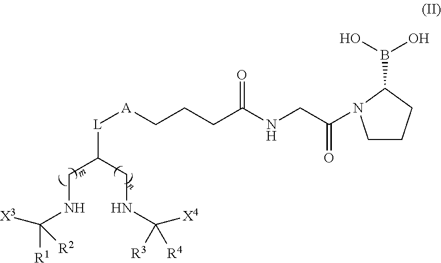
wherein:
A is —(CH₂)ₒ—C(═O)—NH— or —(CH₂)ₚ—NH—C(═O)— wherein each of o and p is an integer between 0-4;
L is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl; and,
X³ and X⁴ are either both —CH₂—NH₂ or both —C(CH₃)═N—OH,
with a suitable source of said metal ion M.

13. A pharmaceutical composition comprising the compound as defined in claim 1 together with a biocompatible carrier suitable for mammalian administration.

14. A kit for carrying out the method as defined in claim 12 wherein said kit comprises the precursor compound of Formula II: 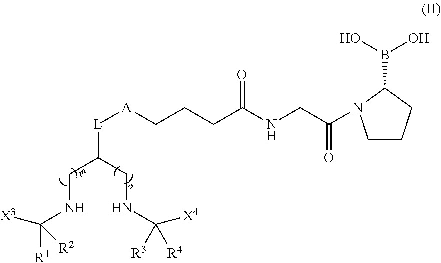
wherein:
A is —(CH₂)ₒ—C(═O)—NH— or —(CH₂)ₚ—NH—C(═O)— wherein each of o and p is an integer between 0-4;
L is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl; and,
X³ and X⁴ are either both —CH₂—NH₂ or both —C(CH₃)═N—OH.

15. An in vivo imaging method comprising:
(i) administering to a subject the compound as defined in claim 1;
(ii) allowing said compound to bind to a biological target in said subject;
(iii) detecting by an in vivo imaging procedure signals emitted by the metal ion of said compound;
(iv) generating an image representative of the location and/or amount of said signals.

16. The in vivo imaging method as defined in claim 15 wherein said compound is administered as a pharmaceutical composition, the pharmaceutical composition comprising the compound of Formula I: 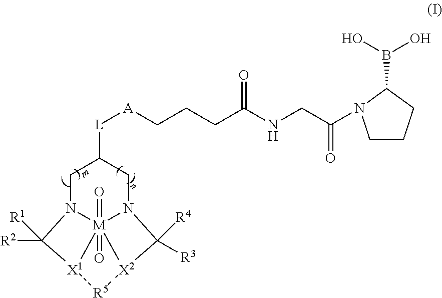
or a salt or solvate thereof;
wherein:
A is —(CH₂)ₒ—C(═O)—NH— or —(CH₂)ₚ—NH—C(═O)— wherein each of o and p is an integer between 0-4;
L is a bivalent linker group having 1-50 bivalent linker units selected from an amino acid residue, a carbohydrate residue, —C(OH)—, —(CR′₂)—, —C(═O)—(CR′2)—, —C(═O)—NR′—, —(CR′₂—O—CR′2)—, —CR′₂—NR′—, CR′₂—S(O₂)—CR′₂, —(CR′₂)—O—N═CR′—, wherein R′ is hydrogen or C₁₋₄ alkyl;
m and n are either both 1 or both 2;
R¹⁻⁴ are either all hydrogen or all methyl;
M is a metal ion selected from ^{99m}Tc, ¹⁸⁶Re and ¹⁸⁸Re; and,
either:
X¹ and X² are both —CH₂—NH wherein each N is co-ordinated to M and R⁵ is not present; or,
—X¹—R⁵—X²— is —C(CH₃)═N—O—H—O—N═C(CH₃)— wherein each N is co-ordinated to M; and
together with a biocompatible carrier suitable for mammalian administration.

17. The in vivo imaging method as defined in claim 15 wherein said biological target is fibroblast activation protein (FAP).

18. The in vivo imaging method as defined in claim 17 which comprises the subsequent step (v) of determining the distribution and extent of FAP expression in said subject wherein said expression is directly correlated with said signals.

19. The in vivo imaging method as defined in claim 15 which is carried out repeatedly during the course of a treatment regimen for said subject.

20. A method for the diagnosis of a condition in which FAP is upregulated wherein said method comprises the in vivo imaging method as defined in claim 18 together with the subsequent step (vi) of attributing the distribution and extent of FAP expression to a particular clinical condition.

21. The method as defined in claim 20 wherein said condition comprises fibrosis.

22. The method as defined in claim 21 wherein said condition is liver fibrosis, congestive heart failure, atherosclerosis or vulnerable plaque.
